(19) United States
(12) Patent Application Publication
(10) Pub. No.: US 2015/0265794 A1
(43) Pub. Date: Sep. 24, 2015

(54) Customized Breathing Mask

(71) Applicant: CPG MEDICAL, B.V., Capelle a.d. IJssel (NL)

(72) Inventors: Cornelis De Kruyff, Klaaswaal (NL); Willem Paul Eduard Eikelenboom, Oud-Beijerland (NL); Govert Machiel De Bruijn, Andel (NL)

(21) Appl. No.: 14/442,102

(22) PCT Filed: Nov. 13, 2013
(86) PCT No.: PCT/EP2013/003423

§ 371 Date: May 15, 2015

(51) Int. Cl. A61M 16/06; A61M 16/00; A61M 11/06

(52) U.S. Cl. CPC A61M 16/0616; A61M 16/0683; A61M 11/065; A61M 16/0003; A61M 2016/0027; A61M 2016/003; A61M 2205/3334; A61M 2205/3368; B33Y 10/00

(57) ABSTRACT

The present invention relates to a manufacturing method and a device for use in breathing assistance systems. The invention provides a cost efficient method for manufacturing a Positive Airway Pressure mask which has an improved fit factor and provides an improved comfort in wearing and a saver use. The mask is produced using 3D rapid prototyping technology using a 3D scan of a subject's face as input. A sealing edge is integrated in the one piece mask.

### CROSS-REFERENCE TO RELATED APPLICATIONS

The present invention claims the benefits of priority from Netherlands patent application NL1039895, filed Nov. 13, 2012, the disclosure of which is incorporated herein by reference.

### TECHNICAL FIELD

This invention relates generally to the field of breathing assistance systems, i.e. systems and methods for providing custom masks for use in a breathing assistance system.

### BACKGROUND OF THE INVENTION

Currently, Positive Airway Pressure (PAP) is a mode of respiratory ventilation used primarily in the treatment of sleep apnea, for which it was first developed. PAP ventilation is also commonly used for those who are critically ill in hospital with respiratory failure, and in newborn infants (neonates). In these patients, PAP ventilation can prevent the need for tracheal intubation, or allow earlier extubation. Sometimes patients with neuromuscular diseases use this variety of ventilation as well. PAP is realized with a nose or face mask placed on the subject during sleep. Through the mask, positive pressure air is continuously delivered to the patient (hereinafter referred to as “subject”). The positive pressure air may be delivered to the subject's upper airway to prevent the upper airway tissues from collapsing during sleep, thus reducing the occurrence and/or severity of sleep apnea. The PAP therapy is also useful in case of poor respiratory function. The breathing assistance system is often used to assist breathing when the subject is asleep, but it may also be used during a wake state, or while being involved in daily activities.

There are various modes of PAP therapy, of which the following modes are described in short.
Continuous Positive Airway Pressure (CPAP)
Bilevel Positive Airway Pressure (BPAP)
Inspiratory Positive Airway Pressure (IPAP)
Lower Expiratory Positive Airway Pressure (EPAP)

Continuous positive airway pressure (CPAP) is the use of continuous positive pressure to maintain a continuous level of positive airway pressure. A ventilator is used, which does not cycle during CPAP. No additional pressure above the level of CPAP is provided, and patients must initiate all of their breaths. Nasal CPAP is frequently used in neonates.

As a treatment or therapy, CPAP uses mild air pressure to keep an airway open. CPAP typically is used for people who have breathing problems, such as sleep apnea. CPAP also may be used to treat preterm infants whose lungs have not yet fully developed. For example, physicians may use CPAP to treat infants who have respiratory distress syndrome or bronchopulmonary dysplasia. In some preterm infants whose lungs haven't fully developed, CPAP improves survival and decreases the need for steroid treatment for their lungs.

CPAP at home utilizes machines specifically designed to deliver a constant flow or pressure. Some CPAP machines have other features as well, such as heated humidifiers. CPAP is the most effective treatment for obstructive sleep apnea, in which the mild pressure from CPAP prevents the airway from collapsing or becoming blocked.

Bilevel Positive Airway Pressure (BPAP) is a CPAP mode used during noninvasive positive pressure ventilation. It delivers a preset inspiratory positive airway pressure (IPAP) and expiratory positive airway pressure (EPAP). BPAP can be described as a CPAP system with a time-cycled or flow-cycled change of the applied CPAP level. CPAP, BPAP and other non-invasive ventilation modes have been shown to be effective management tools for chronic obstructive pulmonary disease and acute respiratory failure. BPAP, is also known as non-invasive positive pressure ventilation (NIPPV) or non-invasive ventilation (NIV), and is sometimes called Variable Positive Airway Pressure (VPAP).

Hereinafter all modes and machines based on positive airway pressure modes will be referred to as PAP, unless otherwise stated.

A major issue with PAP is non-compliance. Studies showed that some users either abandon the use of PAP, and/or use PAP for only a fraction of the nights. Prospective PAP candidates are often reluctant to use this therapy, since the mask feels uncomfortable and has other disadvantages. These disadvantages of conventional masks are mainly caused by a too low Fit Factor (FF) of the mask, wherein a higher FF indicates better fit between the face and the mask and less leakage of air. For example, a low Fit Factor of conventional masks may be a result of many mask assemblies being conform to only standard sizes and shapes. Second, conventional masks may not account for differences in the sizes, shapes (such as facial irregularities) of the facial features of different subjects, thus causing discomfort, pain or leakage of air. Third, because of the poor seals or fits often associated with conventional masks, the mask may not stay in place, and may shift or move. If the mask does not stay in place, or if the mask does not fit closely to the face, air may leak and the air pressure will drop, thereby nullifying the aimed effect of positive pressure.

One way to improve the fit factor is to measure or copy the facial features of a subject more precisely and to produce a customized mask, especially made for the subject. One may make a plaster model of the face, for example, and produce a mask using the created moulds. This requires a fair amount of effort and time, and is therefore costly. It also causes a lot of discomfort and for children and in particular newborns, this is almost impossible to do. An improvement of recent years is to use technologies to improve the capturing of the face, as well as the manufacturing of the mask. Capturing may be done contactlessly by one or more video cameras, or 3d laser scanning for example, whilst manufacturing may be done by using the file with the scanned image as input for a rapid prototyping machine. In this way, the burden for the subject is lessened and the speed and costs of manufacturing are lowered. Using these technologies it is possible nowadays to manufacture CPAP masks or just customized cushions for a CPAP mask within a day.

Rapid prototyping techniques to manufacture customized mask are for example Objet's PolyJet and Shape Deposition Manufacturing (SDM), as disclosed in e.g. Yih-Lin Cheng, Jin-Chiou Chu, (2012),“Application of Rapid Tooling to Manufacture Customized Nasal Mask Cushion for Continuous Positive Airway Pressure (CPAP) Devices”, Rapid Prototyping Journal, Vol. 19 Iss: 1 (Date online Aug. 7, 2012).

United States patent application, U.S. 2008078396 A1, by Nellcor Puitan Bennett Inc, as published on Apr. 3, 2008 discloses a method for fabricating a facial seal for use in a breathing assistance system. The method described therein includes capturing one or more images of a subject's face. The method further includes translating the one or more images into a set of data representing a three-dimensional structure of at least a portion of the subject's face and fabricating a facial seal to substantially conform to the subject's face based at least on the set of data, including fabricating at least a portion of the facial seal using rapid prototyping.

Although surface scanning of an individual subject leads to a quite accurate capturing of the subject's face surface, and although the captured image may lead to a better fit factor when using rapid prototyping technologies, the comfort for the subject in wearing the mask may often still be insufficient, or even worse than masks produced in an old fashioned manner using a plaster mould and a lot of craftsmanship. The main reason for this discomfort is that the mask produced by rapid prototyping follows the contours of the subject's face in a situation wherein there is no mask placed on the face (relaxed state). In practical use, the mask usually exerts a pressure on the facial tissue and therefore deforming the face. A mask based on capturing a face in relaxed state therefor may produce sharp edges which are in contact with the subject's face. This may feel uncomfortable for the subject when the mask is pressed tightly on the subject's face by fastening the mask around the subject's head with for example elastic straps. To limit this discomfort, usually some kind of extra flexible soft seals may be applied between the mask and the subject's face. These seals usually increase the fit factor and the comfort for the subject.

The extra seal has the disadvantage that it results in a mask with more complexity, and an extra point of failure. A further disadvantage is that every extra joint between parts of the mask is a possible area for accumulation of dirt and bacteria, which may cause sanitary problems.

### BRIEF SUMMARY OF THE INVENTION

It is an object of the invention to provide a cost efficient method for manufacturing a mask, preferably a Positive Airway Pressure mask which has an improved fit factor, is hygienic in use because of its construction, and provides an improved comfort in wearing and a saver use. Furthermore it is an object of the invention to provide a PAP mask, providing these objectives.

The invention realizes the objectives in the following manner. Firstly a subject's face is captured, using 3D capturing technology such as 3D cameras. The captured image is input for a rapid prototyping manufacturing process. The PAP mask is then manufactured in one production run (in a continuous process). With modern rapid prototyping technologies, and especially by applying additive manufacturing or 3D printing, it is possible to change types of material during the printing process. This provides the surprisingly effective possibility to integrate a seal into the edge of the mask, instead of manufacturing a separate cap and a separate seal, as currently is the state of the art technology. In this way the seal and the cap of the mask (the cap being the mask part without the seal), are seamlessly joined. This saves one assembly step, which in turn saves costs on the one hand, and prevents possible leakage because of a bad connection between the seal and the cap on the other hand.

By using computer aided 3D capturing of the face, in combination with 3D manufacturing of the mask, an accurate mask can be custom produced. The edge of the mask will fit extremely well to the contours of the face, and because of the possibility to customize the mask, the mask can be produced in virtually every shape necessary to achieve a near perfect fitting form for each subject. This is especially advantageous, taking into account that no face is the same; moreover a lot of faces show irregularities, such as asymmetries.

Thus a very good fit factor is achieved, which leads to an increased comfort in wearing and at least as important, a saver use, because of considerable less possibility of air leakage. Moreover, because of the seamless integration of a seal, there is less chance of bacteria and dirt to collect in cavities, therefore a sanitary improvement is achieved.

In order to increase comfort and safety and to lower production costs, the 3D manufacturing process allows incorporating attachment means for supplementary devices, according to the needs or wishes of the subject, which increase either comfort or safety. One may think of a sensor which detects if a leakage occurs, despite of the good fit factor. The subject may for example turn in his sleep and the mask may get loose accidentally, without the subject noticing it. Such a sensor will be able to detect this leak and gives a warning signal.

These example embodiments and further embodiments of the present invention, including their advantages will be described hereinafter in detail.

BRIEF DESCRIPTION OF THE SEVERAL VIEWS OF THE DRAWINGS

In order that the invention may be more readily understood, and so that further features thereof may be appreciated, the invention will now be described, by way of example, with reference to the accompanying drawings wherein like numbers refer to same and like parts, wherein:

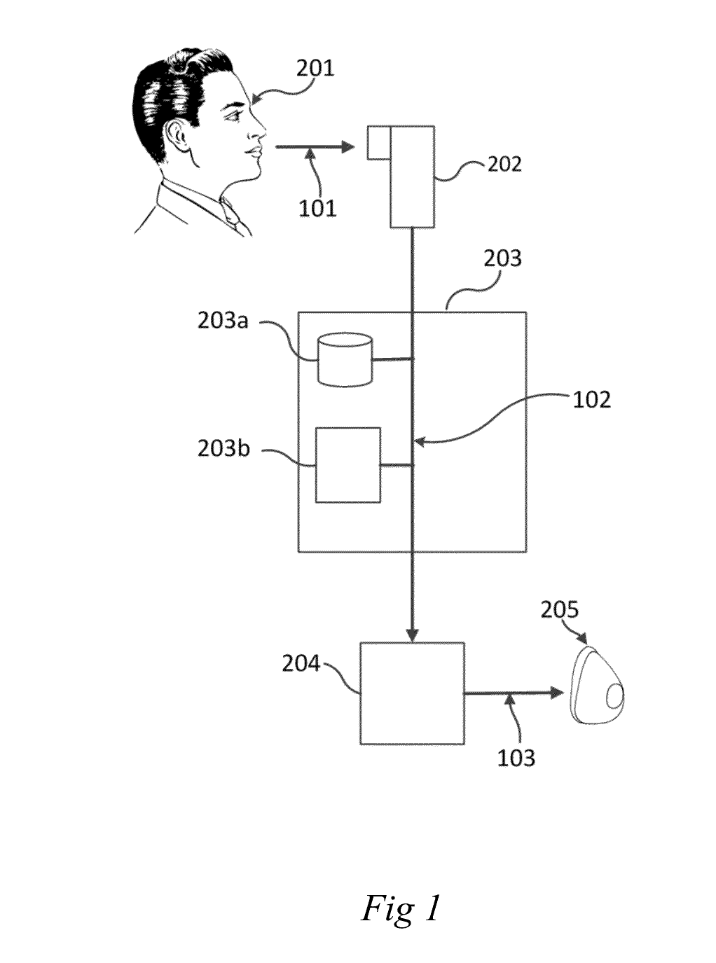

FIG. 1 shows the method of the invention.

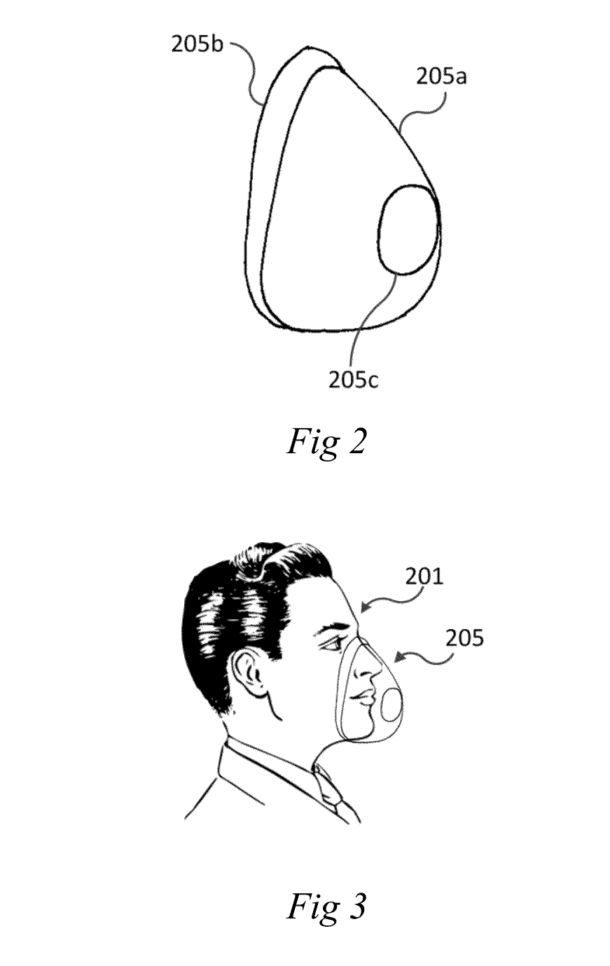

FIG. 2 shows a customized mask according to the invention.

FIG. 3 shows a subject wearing the customized mask according to the invention.

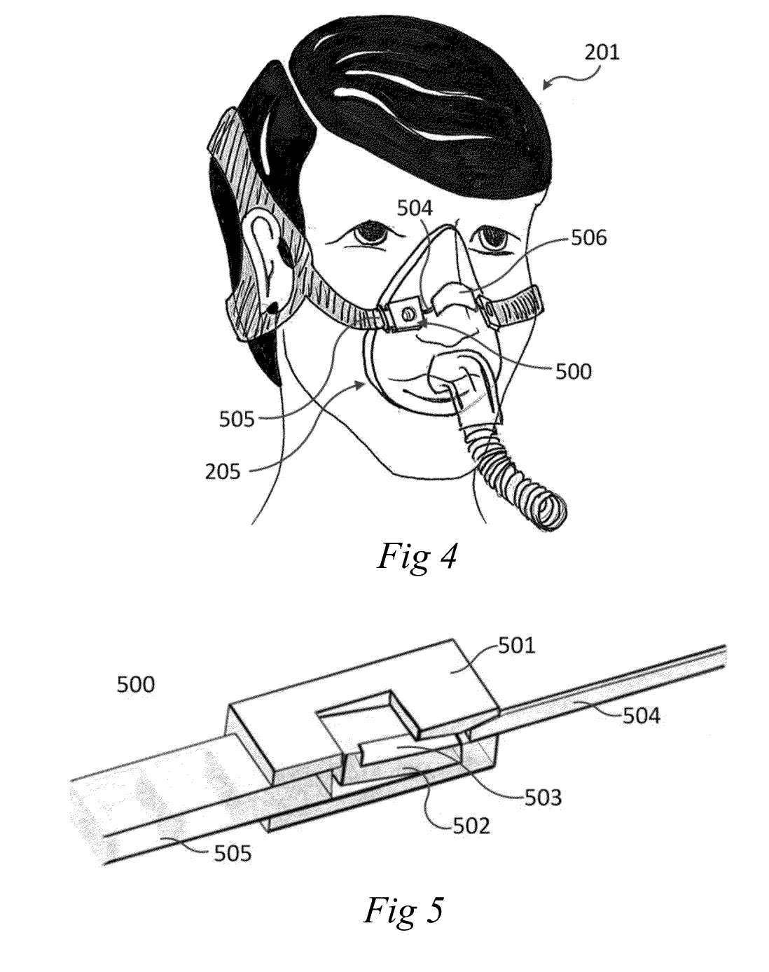

FIG. 4 shows a customized mask with a quick release mechanism.

FIG. 5 shows a detail of a cross-section of a quick release mechanism.

### DETAILED DESCRIPTION OF THE INVENTION

Referring now to FIG. 1, a method in accordance with the invention is shown, wherein the face of a subject 201 is scanned with a 3D scanner 202. A computer 203 translates the images into data for a manufacturing machine. For this purpose the computer 203 comprises a storage device 203a for storing the captured images and software running on processor/translator unit 203b for translating 102 the images into a set of data representing a three-dimensional structure of at least a portion of the face. The data can be read by a manufacturing machine 204, preferably a rapid prototyping machine. The manufacturing machine 204 fabricates 103, based on the set of data, at least part of the customized mask 205 using additive manufacturing technology. At least part of the customized mask 205 is fabricated in one piece such that at least a portion of the cap 205a and at least a portion of the edge 205b (see FIG. 2) are seamlessly joined in the same production run, or are made to be assembled afterwards and fit together with very high precision, preferably with a tolerance of less than 1 mm, more preferably with a tolerance of less than 0.5 mm. At least a portion of the edge 205b is fabricated using material which is more resilient than the material used for the at least a portion of the cap 205a. Fabrication is preferably done using a 3D printer, using photopolymerization and applying Digital Light Processing (DLP) technology. Other suitable technologies may stereolithography (STL) and selective laser sintering (SLS).

Referring now to FIG. 2, a customized mask 205 in accordance with the invention is shown. The customized mask 205 may be used with a breathing assistance system for supplying a flow of air to a subject. The mask comprises a cap 205a arranged for covering at least part of a face of a subject and an edge 205b arranged for making contact to at least part of the face. The mask 205 is custom fabricated to fit the face of the subject 201, wherein the edge 205b substantially follows the contours of the face. To distribute the pressure that the mask 205 exerts on the face when the subject 201 wears the mask 205, the edge 205b may be made of a resilient material. In order to maintain the form of the cap 205a, the cap 205a may be made of less resilient material in comparison with the material of the edge 205b. Rapid prototyping technology allows applying of materials which differ in characteristics such as resilience and transparency. The mask 205 may be produced in a single production run, without a need for assembling the edge 205b and the cap 205a in a separate manufacturing step. The resilient edge 205b may be thicker at the location where the edge 205b makes contact with the relative harder parts of the face of the subject 201. The resilient edge 205b may then be thinner at the location of the softer parts of the face. In this way the pressure of the mask 205 on the face will be distributed even better along the edge 205b. In the same production run where the mask is made, an arrangement 205 may be made in the mask 205 to attach a tube for transporting air from a breathing assistance system. The arrangement 205 comprises an aperture to facilitate intake of air through the tube.

Referring now to FIG. 3, a subject 201 wearing the customized mask 205 is shown in order to depict how the mask may be worn.

Referring now to FIG. 4, a subject 201 is shown wearing the customized mask 205 which is fastened around the subject's head with straps 505. The mask comprises attachment means for the straps. The attachment means in this exemplary embodiment is of the type of a quick release device 500, which may be opened with a single movement. Until now it has been considered a problem that, in case of emergencies or in case of limited use of both hands, no solution was available to provide a cheap and simple closing and opening mechanism. Now that this invention provides for a one run production process, the mask may be produced with an incorporated quick release system, which does not need assembly. This prevents substantially unsafe positioning of the closure system and a solid attachment and provides a cost efficient solution. With a single hand movement the strap may be released and the mask falls off. This enables doctors for example to initiate a medical procedure on the head of the patient such as an emergency intubation more easily, without a need to move the head of the subject for releasing of the straps. The release mechanism is on the mask after all.

Referring now to FIG. 5, a cross-section of a quick release device is shown with a housing 501, arranged for inserting the strap (505). The strap's end has a latch 502, which is retained by the housing 501. In order to release the latch, push-in rod 504 is inserted in recess (503) of the latch 502. The push-in rod 504 pushes away (which is in a downward direction in FIG. 5) latch 502 in order for the strap 505 to be removed to the left from the housing 501. The push-in rod is operated with bridge like button 506. If the subject or a third person intents to quickly release the mask, one has to press the bridge like button 506. By pressing this button 506, the push-in rod 504 is moved sideways and further into the housings 501. This operates the releasing mechanism. This configuration allows for a simultaneous operation of a second latch and housing on the other side of the mask as shown in the figure. It is obvious that also more than two latch housing arrangements may be operated simultaneously. Vice versa, in order to attach strap 505 to the housing 501 the strap 505 needs to be inserted in the housings 501. In this way strap 505 is attached on one side of the mask 205, whereas preferably another strap may be attached at the other side of the mask 205. The release mechanism may of course also be attached to any mask after production of the mask.

The invention is now described by the following aspects and embodiments.

In a first aspect of the invention a method is provided for fabricating a customized mask for use with a breathing assistance system for supplying a flow of air to a subject, the customized mask comprising a cap arranged for covering at least part of the face of a subject and an edge arranged for making contact to at least portion of the face, the method comprising:
capturing of one or more images of at least a portion of the face;
translating the one or more images into a set of data representing a three-dimensional structure of at least a portion of the face; and
fabricating, based on the set of data, at least part of the customized mask using additive manufacturing technology,
   characterized in at least a portion of the cap and at least a portion of the edge are seamlessly joined, wherein the at least a portion of the edge is fabricated using material which is more resilient than the material used for the at least a portion of the cap.

By using material for the edge which is more resilient than the cap material, the edge will function as a seal. The flexibility is needed to adjust the seal to small irregularities of the face's skin, which may occur for example while moving the face, talking, or lying the face to the side on a pillow. The resilience of the edge increases the comfort of wearing of the mask for the subject, because of an improved distribution of force of the mask on the face. A rounded edge or an edge with increased contact surface will improve distribution of force even better. The 3D manufacturing process allows for adjusting the edge in shape, size and resilience, which provides many possibilities to adjust the mask to the personal preferences of the subject. Because the cap and the edge of the mask are produced for the purpose of fitting together seamlessly, there is no possibility of air leakage because of a bad connection between cap and edge. This increases safe use. Moreover this improves hygiene, because dirt and microbes have less chance to get stuck in cavities. This also improves the easiness of cleaning of the mask.

In a first embodiment of the invention, the capturing of one or more images comprises capturing of one or more images of at least a portion the face which is in a substantially horizontal upward facing position. By capturing the face while the subject is lying on his back, the face may assume a position which is favourable when the subject lies asleep. The face will deform such that soft facial tissue will be indented because of gravity. By consequence the shape of the mask will have an improved fit factor, especially in the situation wherein the subject is asleep, lying on his back. If the subject is upright however, the shape of the mask will be such that the contour of the edge will provide an extra force on the softer parts of the face. This will increase the fit factor even more, because of an improved distribution of force along the edge.

In a second embodiment, an additional step is performed before capturing of one or more images, the additional step comprising applying an indentation force on at least a portion of the face. Instead of or in addition to capturing the face in a horizontal position, the face may be deliberately and targeted impressed by a mechanical force. The indentation force may be provided in the simplest form by pushing fingers into the softer parts of the face, or by applying a spring load to specific parts of the face. Also a transparent object may be used to apply the force. In the computer processing of the captured images, it is possible to erase the devices or hand which has applied the indentation force if necessary. The advantage of having a captured indented face is that the mask can be printed such that it will exert more force on the softer parts of a relaxed face. In turn this will lead to a more even distribution of force of the mask on the face. Especially the less resilient parts of the face such as the location of the cheekbones or the bony part of the nose will experience less pressure, which increases comfort of wearing.

In a third embodiment, capturing of one or more images of the face of the subject comprises using an image capturing technique of the group comprising:

Photometric scanning, such as system using a single camera taking multiple images;
laser scanning;
stereo photography;
tactile imaging.

These capturing techniques provide images which are accurate, quick and easy to make, and with minimum discomfort for a subject. By using these techniques, no plaster is needed anymore. Plaster is considered very uncomfortable, because of coverage of parts of the face and the relatively long duration of this multi-stage procedure. The images captured with these techniques are easy to use in computer aided manufacturing processes and in particular in rapid prototyping processes. Especially photometric scanning is effective because it requires no laser emitted at the subject, or touching of the subject. By moving the video camera around the subject, an accurate 3D image can be reproduced using advanced 3d software. An example product is the Artec 3D Scanner, which comprise a single video camera which captures separate frames, together forming a 3D image. The concept of this scanning process is fairly simple: Multiple images are captured by moving the scanner around the subject, or by turning the subject in front of the camera. The latter option, of course would be less favourable considering the comfort of the subject. The separate images are combined into a single 3D mesh by scanning software which recognizes the unique geometry of each captures image.

In a fourth embodiment, the additive manufacturing technology comprises one of the group comprising:
Powder bed and inkjet head 3d printing
Shape Deposition Manufacturing
Fused Depositioning Modeling
Fused Filament Fabrication (FFF)

These additive manufacturing techniques allow for real time varying of material in the same continuous production process, with seamless joints. More particular it allows for using resilient material for the edge of the mask. In this way the edge functions as flexible seal. By avoiding the need for a follow up process step of assembly of the seal to the cap, the earlier mentioned hygiene, comfort and production costs advantages are achieved.

In a fifth embodiment the at least part of the customized mask 205 is fabricated in one piece such that at least a portion of the cap 205a and at least a portion of the edge 205b are seamlessly joined. Additive manufacturing allows for using different materials in the same production run. The edge may therefore be produced together with the cap as one piece, while the edge 205b may be made of a more resilient material than the cap 205a. In this way an even more efficient production method is achieved. At the same time no assembly of cap and edge is necessary and no joint exists where microbes and dirt may settle. This prevents also leakage of air between cap and edge even better. This leads to a safer use, better cost efficiency and better hygiene.

In a second aspect of the present invention, a customized mask is provided for use with a breathing assistance system for supplying a flow of air to a subject, comprising a cap arranged for covering at least part of a face of a subject and an edge arranged for making contact to at least part of the face, characterized in that the mask is custom fabricated to fit the face of a subject, wherein the edge substantially follows the contours of the face. In this way a mask is provided which suits an individual subject, taking into account most, if not all, irregularities of the subject's face. This increases the fit factor, which in turn increases comfort in wearing and air tightness along the edge.

In a first embodiment of the second aspect of the invention, the contours of the face comprise the contours of the face in an at least partly substantially relaxed state. This provides the simplest solution with the least discomfort for the subject while capturing the face.

In a second embodiment of the second aspect, the contours of the face comprise the contours of the face in an at least partially indented state, which indented state is relative to the relaxed state. This will lead to a better distribution of force of the edge of the mask exerted on the face. The contour of the edge will follow the indented face, which means that when the mask is worn, the edge will exert more force to the softer parts of the face in comparison with a contour of the edge which follows the contours of a relaxed face.

In a third embodiment of the second aspect, at least an area of the cap protrudes extra towards the face at a location of a relatively more resilient part of the at least one portion of the face, and the edge is proportionally thinner at the location of said area of the cap. When the cap is constructed such that it protrudes towards a more resilient part of the face, the force exerted by the mask on that part will increase as described in the second embodiment of the second aspect. By taking an extra measurement, namely decreasing the size of the edge at the area of the extra protrusions, the resulting contour of the edge is less protruding. It is even possible to fully compensate the extra protrusion with a thinner edge, in order to follow the contours of a relaxed face. This has the advantage that the relaxed state of the face may be taken as starting point for capturing of the images of the face, whilst at the same time a built-in mechanism in the customized mask provides a more even distribution of forces of the mask on the face. This mechanism works as follows. At the areas of the edge where the edge is thinner there is less resilient material (measured perpendicular to the direction of the indentation force of the mask). This means that the force on the softer parts of the face is increased, whereas the force on the less resilient parts of the face is decreased, because of relatively more resilient material at that area of the edge. In general this means that the experienced force on for example the cheek bones and bony part of the nose is less, which leads to increased comfort in wearing and less chance of air leakage.

In a fourth embodiment of the second aspect the mask comprises an attachment means arranged for attachment of a supplementary device. Many subjects have special needs or wishes. The customized mask provides possibilities to incorporate means into the product during a single 3D production run, in very complicated structures and shapes. The invention makes it possible to position these means at the optimal locations for each individual, taking into account irregularities of the face. The devices will increase either safety, comfort or both.

In a fifth embodiment of the second aspect, the attachment means comprises a recess or aperture arranged for insertion of the supplementary device. In this way a device may be inserted into a part of the mask. The device does not have to be adjusted to the attachment means of the mask, because the 3D printing technique allows customizing the attachment means instead, in particular recesses or apertures can be dimensioned to hold a particular device.

In a sixth embodiment of the second aspect, the supplementary device comprises a sensor of the group comprising:
A carbon dioxide sensor arranged for measuring the amount of carbon dioxide in the air of the flow of air;
A temperature sensor arranged for measuring temperature of the air of the flow of air;
An airflow meter arranged for measuring the speed of flow of air through the mask;
A pressure sensor or a force sensing resistor arranged for measuring pressure of the mask on the face;
A humidity sensor arranged for measuring humidity of the air of the flow of air;
An audio sensor or microphone arranged for measuring sound.

By attaching these example sensors to the mask, the safety of use is increased. A carbon sensor can measure the quality of inhaled or exhaled air. If the level of Carbon Dioxide is too high or too low, a warning signal may be given or an adjustment to the breathing assistance system may be initiated. A temperature sensor can measure the temperature of the air. In this way the inhaled air for example can be controlled. Too cold air is very uncomfortable for a subject to inhale, as is too hot air. An airflow meter positioned at risk areas of the mask, where there is a risk of air leakage, for example near the corners of the mouth of the subject. A pressure sensor near or on the edge of the mask can measure pressure exerted by the mask on the faced. This makes it possible to give a warning signal when the pressure is below a certain threshold, indicating that a possible air leakage may occur. A humidity sensor measures humidity of the inhaled air. In this way, a signal could be send to the breathing assistance system whenever humidity is too low. Often these systems are equipped with air humidifiers which can control the amount of vaporized water in the air submitted to the subject. An audio sensor can register changes in sound, such as pitch of breathing sounds. When irregularities are measured, a warning system can be activated.

In a seventh embedment of the second aspect, the supplementary device comprises an injector arranged for injection of a substance such as water, in the form of steam, liquid, or aerosol, into the mask. As mentioned in the previous embodiment, a breathing system can be equipped with a humidifier. Instead of or in addition to providing humidified air from the system, water (in either form) can be applied through the mask. This has the big advantage, that there is less or no need for a complicated humidifier in the breathing assistance apparatus. Another advantage is that the water is applied much closer to the mouth of the subject, which leads to a better control and a more direct feedback loop. Current systems have to take into account the length of the tube, external circumstances, heat loss in the tube etcetera. This is not relevant if the water is applied directly near the mouth.

In an eighth embodiment of the second aspect, a control is provided, arranged for the operation of the injector. By adding a control for the injector, the subject can control the operation of the injector according to his personal preferences in a very direct manner. This increases confidence of the subject and comfort in operation.

In a ninth embodiment of the second aspect, the control is arranged to be manipulated by the tongue of the subject wearing the mask. By positioning the control of the injector in the mask near the mouth, the subject can manipulate the control with his tongue. Water (in any form) can be injected directly into his mouth or added to the air that he inhales. Especially in cases wherein the subject has limited control of his hands, often the tongue is perfectly able to manipulate a control. It also provides freedom of the hands for daily tasks for example, without having to stop the task for adjustment of the control with a hand, thus providing hands-free operation.

In a tenth embodiment of the second aspect, the supplementary device comprises a strap. The customization of the mask provides a lot of freedom of design of the shape, function and position of attachments for one or more straps. These straps are usually made of elastic material and fasten the mask to the face of the subject. By careful design of the attachment means, the working of the straps and the comfort of the straps can be optimized, thus leading to more safety and comfort.

In an eleventh embodiment the attachment means comprises a releasing device (500) arranged for releasing the strap (505) with a single movement.

In a twelfth embodiment the releasing device (500) comprises a housing (501), arranged for inserting the strap (505), the strap (505) comprising a latch (502) arranged to be retained by the housing (501) and arranged to be released by insertion of a push-in rod (504) in a recess (503) of the latch (502).

In a thirteenth embodiment the edge of the customized mask comprises a flange arranged for attachment of an additional seal.

In this way, the edge may be manufactured as a thin slab, in the range of a few millimetres width. The edge would, however, possibly notch the face in that case, which would feel uncomfortable. Additionally or as replacement of the resilience of the edge, a flange may be provided on the edge, which increases the contact area, as such, but additionally allows attaching an additional seal to the edge. This seal may be made of resilient material and have sufficient width in order not to notch the face of the subject. This will increase comfort for the subject and prevents mask air leakage, thus leading to a safer use.

In a fourteenth embodiment the customized mask comprises a customized Positive Airway Pressure (PAP) mask. The mask as discussed can be a PAP mask. The advantages of the present invention as mentioned above are particular important for a PAP mask. Air leakage and discomfort are typical aspects of prior art PAP masks and are dealt with in this invention.

The term “substantially” herein, such as in “substantially horizontal” etc., will be understood by the person skilled in the art. In embodiments the adjective substantially may be removed. Where applicable, the term “substantially” may also include embodiments with “entirely”, “completely”, “all”, etc. Where applicable, the term “substantially” may also relate to 90% or higher, such as 95% or higher, especially 99% or higher, including 100%. The term “comprise” includes also embodiments wherein the term “comprises” means “consists of.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The term “and/or” includes any and all combinations of one or more of the associated listed items. The article “a” or “an” preceding an element does not exclude the presence of a plurality of such elements. The article “the” preceding an element does not exclude the presence of a plurality of such elements. In the device claim enumerating several means, several of these means may be embodied by one and the same item of hardware. The mere fact that certain measures are recited in mutually different dependent claims does not indicate that a combination of these measures cannot be used to advantage.

### BEST MODE FOR CARRYING OUT THE INVENTION

The best mode for carrying out the invention would be a method for fabricating a customized PAP mask, comprising a cap arranged for covering part of the face of a subject and an edge arranged for making contact to the face, wherein the mask is fabricated such that the cap and the edge are seamlessly joined, wherein the edge is fabricated using material which is more resilient than the material used for the cap. The mask further comprises an attachment means arranged for attachment of a supplementary device. The method further comprises:
capturing of one or more images of at least a portion of the face which is in a substantially horizontal upward facing position, wherein the capturing is done using Photometric scanning, with a single camera taking multiple images;
translating the one or more images into a set of data representing a three-dimensional structure of at least a portion of the face;
fabricating, based on the set of data, the mask using rapid prototyping manufacturing technology, applying additive manufacturing technology.

1. A method for fabricating a customized mask (205) for use with a breathing assistance system for supplying a flow of air to a subject (201), the customized mask comprising a cap (205a) arranged for covering at least part of the face of the subject (201) and an edge (205b) arranged for making contact to at least portion of the face, the method comprising:
capturing (101) of one or more images of at least a portion of the face;
translating (102) the one or more images into a set of data representing a three-dimensional structure of at least a portion of the face; and
fabricating (103), based on the set of data, at least a part of the customized mask (205) using additive manufacturing technology,
characterized in that,
at least a portion of the cap (205a) and at least a portion of the edge (205b) are seamlessly joined, wherein the at least a portion of the edge (205a) is fabricated using material which is more resilient than the material used for the at least a portion of the cap (205b).

2. A method for fabricating a customized mask (205) according to claim 1, characterized in that the capturing (101) of one or more images, comprises capturing of one or more images of at least a portion the face which is in a substantially horizontal upward facing position.

3. A method for fabricating a customized mask (205) according to claim 1, characterized in that an additional step is performed before capturing (101) of one or more images, the additional step comprising applying an indentation force on at least a portion of the face.

4. A method for fabricating a customized mask (205) according to any one of the claims 1-3, characterized in that the capturing (101) of one or more images comprises using an image capturing technique of the group comprising:
Photometric scanning, such as a system using a single camera taking multiple images;
laser scanning;
stereo photography;
tactile imaging.

5. A method for fabricating a customized mask (205) according to any one of the preceding claims, characterized in that the additive manufacturing technology comprises one of the group comprising:
Powder bed and inkjet head 3d printing
Shape Deposition Manufacturing
Fused Depositioning Modeling
Fused Filament Fabrication (FFF)

6. A method for fabricating a customized mask (205) according to any one of the preceding claims, characterized in that the at least part of the customized mask (205) is fabricated in one piece such that at least a portion of the cap (205a) and at least a portion of the edge (205b) are seamlessly joined.

7. A customized mask (205) for use with a breathing assistance system for supplying a flow of air to a subject (201), comprising a cap (205a) arranged for covering at least part of the face of the subject (201) and an edge (205b) arranged for making contact to at least part of the face,
characterized in that,
the mask (205) is custom fabricated to fit the face of a subject (201), wherein the edge (205b) substantially follows the contours of the face.

8. A customized mask (205) according to claim 7, characterized in that the contours of the face comprise the contours of the face in an at least partly substantially relaxed state.

9. A customized mask (205) according to any one of the claims 7-8, characterized in that the contours of the face comprise the contours of the face in an at least partially indented state, which indented state is relative to the relaxed state.

10. A customized mask (205) according to any one of the claims 7-9, characterized in that at least an area of the cap (205a) protrudes extra towards the face at a location of a relatively more resilient part of the at least one portion of the face, and the edge (205b) is proportionally thinner at the location of said area of the cap (205a).

11. A customized mask (205) according to any one of the claims 7-10, characterized in that the mask (205) comprises an attachment means arranged for attachment of a supplementary device.

12. A customized mask (205) according to claim 11, characterized in that the attachment means comprises a recess or aperture arranged for insertion of the supplementary device.

13. A customized mask (205) according to any one of the claims 11-12, characterized in that the supplementary device comprises a sensor of the group comprising:
A carbon dioxide sensor arranged for measuring the amount of carbon dioxide in the air of the flow of air;
A temperature sensor arranged for measuring temperature of the air of the flow of air;
An airflow meter arranged for measuring the speed of flow of air through the mask (205);
A pressure sensor or a force sensing resistor arranged for measuring pressure of the mask (205) on the face;
A humidity sensor arranged for measuring humidity of the air of the flow of air;
An audio sensor or microphone arranged for measuring sound.

14. A customized mask (205) according to any one of the claims 11-12, characterized in that the supplementary device comprises an injector arranged for injection of a substance such as water, in the form of steam, liquid, or aerosol, into the mask (205).

15. A customized mask (205) according to claim 14, characterized in that the customized mask (205) comprises a control arranged for the operation of the injector.

16. A customized mask (205) according to claim 15, characterized in that the control is arranged to be manipulated by the tongue of the subject (201) wearing the mask (205).

17. A customized mask (205) according to any one of the claims 11-12, characterized in that the supplementary device comprises a strap (505).

18. A customized mask (205) according to claim 17, characterized in that the attachment means comprises a releasing device (500) arranged for releasing the strap (505) with a single movement.

19. A customized mask (205) according to claim 18, characterized in that the releasing device (500) comprises a housing (501), arranged for inserting the strap (505), the strap (505) comprising a latch (502) arranged to be retained by the housing (501) and arranged to be released by insertion of a push-in rod (504) in a recess (503) of the latch (502).

20. A customized mask (205) according to any one of the claims 7-19, characterized in that the edge (205b) of the customized mask (205) comprises a flange arranged for attachment of an additional seal.

21. A customized mask (205) according to any one of the claims 7-20, characterized in that the customized mask (205) comprises a customized Positive Airway Pressure (PAP) mask.
